(19) United States
(12) Patent Application Publication
(10) Pub. No.: US 2025/0027940 A1
(43) Pub. Date: Jan. 23, 2025

(54) Isolation and Diagnostics of Fetal Cells

(71) Applicant: ARCEDI BIOTECH APS, Vejle (DK)

(72) Inventors: Ripudaman Singh, Aarhus C (DK); Palle Schelde Jensen, Aarhus C (DK)

(21) Appl. No.: 18/714,193

(22) PCT Filed: Dec. 6, 2022
(86) PCT No.: PCT/EP2022/084542

§ 371 Date: May 29, 2024

(30) Foreign Application Priority Data
Dec. 6, 2021 (EP) 21212578.5

(51) Int. Cl. G01N 33/569; G01N 33/68

(52) U.S. Cl. CPC G01N 33/56966; G01N 33/6863; G01N 33/6872

(57) ABSTRACT

The present disclosure relates to methods of fetal cell isolation and methods of prenatal diagnostics. The disclosure furthermore provides novel markers for fetal cell isolation that can be used to isolate fetal cells for prenatal diagnostics.

### TECHNICAL FIELD

The present disclosure relates to isolation and diagnostics of fetal cells from a sample, such as a blood sample, derived from a pregnant woman.

### BACKGROUND

During pregnancy, fetal cells from the placenta can invade the maternal blood circulation. Most of these fetal cells originate from the placenta, and are trophoblasts, endovascular trophoblasts or extravillous trophoblasts (EVTs). Common methods of isolating fetal cells are laborious and limited to a narrow time frame of a few weeks. These common methods include steps to remove plasma and maternal cells from the blood, followed by enrichment of fetal cells using specific antibodies targeting these cells. One such method of enrichment is based on Magnetic Activated Cell Sorting (MACS) where antibodies conjugated to magnetic beads are used to target fetal cells. After enrichment, fetal cells are stained with specific antibodies and identified and isolated using different technologies, including fluorescence scanner integrated with a microscope, or Fluorescence Activated Cell Sorting (FACS). The ideal gestational age of isolating placenta derived fetal cells is around 12 weeks, following which their numbers drop, hampering their use in prenatal testing.

### SUMMARY

The inventors have made the surprising finding that cells of the fetal membranes are shed into maternal circulation during amniogenesis and especially in high numbers when the membranes are expanding. Thus, suitable markers of fetal membrane cells can be used to isolate fetal membrane cells. Since fetal membranes are expanding between weeks 15-20, one advantage of using fetal membrane cell markers is that fetal cells can be isolated in a much longer period after gestation, than is possible using common methods.

Thus, in one aspect, the disclosure relates to a method of isolating fetal membrane cells comprising the steps of:
a. providing a maternal blood sample obtained from a woman carrying a fetus;
b. contacting the cells comprised in the maternal blood sample with a ligand directed against a fetal membrane cell marker;
c. optionally, enriching the maternal blood sample for fetal cells
d. detecting the fetal membrane cell; and
e. isolating the fetal membrane cell.

Having realized the possibility of isolating fetal membrane cells, the inventors furthermore provide a use of these cells for prenatal diagnostics.

Thus, in a further aspect, the disclosure relates to a method of prenatal diagnostics comprising
a. obtaining a fetal cell isolated by a method of the disclosure; and
b. diagnosing the fetus.

### DETAILED DESCRIPTION

The present disclosure relates to methods of enriching and/or isolating fetal cells from maternal samples, such as blood samples. In certain aspects of the disclosure, the fetal cells are fetal membrane cells and are located in the blood stream of the mother carrying the fetus, since the cells are shed into the bloodstream during amniogenesis, and especially in high numbers when the membranes are expanding, this can be particularly exploited to enrich and/or isolate fetal cells. Markers suitable for use in the methods of the invention are described further below. A particular aspect of the disclosure relates to a method of prenatal diagnostics comprising the steps of:
a. providing a maternal blood sample obtained from a woman carrying a fetus;
b. isolating a fetal membrane cell from the maternal blood sample comprising the steps of:
   i. contacting the cells comprised in the maternal blood sample with a ligand directed against a fetal membrane cell marker;
   ii. optionally, enriching the maternal blood sample for fetal cells
   iii. detecting the fetal membrane cell; and
   iv. isolating the fetal membrane cell;
c. diagnosing the fetus.

When the term “fetal membrane cell” is used in the disclosure, it is not meant to be understood as being limiting on the type of fetal cell and can mean any type of fetal cell, such as any type of fetal cell described herein.

Also encompassed by the present disclosure are fetal cells expressing one or more of IGF2, MUC16, UPK1B, EMP1, GPX8, FLT1/VEGFR1, RXFP1, CNR1, PRLR or THY1 and combinations thereof and methods of isolating these.

Further encompassed by the present disclosure are fetal cells expressing one or more of MUC16, UPK1B, EMP1, GPX8, FLT1/VEGFR1, RXFP1, CNR1, PRLR, NPR3/NPR-C, FERMT2 and THY1 and combinations thereof and methods of isolating these.

Additionally encompassed by the present disclosure are fetal cells expressing one or more of IGF2, MUC16, UPK1B, EMP1, GPX8, FLT1/VEGFR1, RXFP1, CNR1, PRLR, NPR3/NPR-C, FERMT2 and THY1 and combinations thereof and methods of isolating these.

### Amniogenesis

Amniogenesis is the process of formation of the fetal membranes and is formed early in development. Fetal membranes are the membranes surrounding the embryo or fetus. These membranes are the amnion, the chorion, the allantois, and the yolk sac. As used herein, the term “chorion” refers to the outermost fetal membrane around the embryo. The chorion consists of two layers: an outer formed by the trophoblast, and an inner formed by the somatic mesoderm; the inner layer is in contact with the amnion. The chorion and the amnion form the amniotic sac which comprises the amniotic fluid. The amnion is a membrane that lies next to the chorion. It is a thin and tough membrane. As used herein, the term “amnion” refers to the membrane that covers the embryo when first formed. It further contains the amniotic fluid and serves to provide a protective environment for the developing embryo or fetus. The fetal membranes are expanding when the fetus grows, and during expansion, a higher number of cells are shed into the circulation. In one embodiment of the disclosure, the fetal membranes are expanding when the maternal blood sample is taken. Typically, fetal membranes are expanding between the gestational ages weeks 15 to 20, thus in one embodiment, the maternal blood sample is taken at gestational ages weeks 15-20.

The gestation period is how long a woman is pregnant. Most babies are born between 38 and 42 weeks of gestation. The actual date of conception is generally not known for humans, so gestational age is the common way to measure how far along a pregnancy is. Gestational age as used herein is measured in weeks from the first day of a woman's last menstrual period.

Since cells are shed into the maternal circulation, a blood sample from a woman carrying a fetus can be used to isolate fetal cells. In some embodiments of the disclosure, a cellular fraction in the maternal blood sample comprises both maternal cells and fetal cells. In some embodiments of the disclosure, fetal cells in general are enriched and isolated by methods of the disclosure. In one embodiment of the disclosure, the cellular fraction comprises red blood cells, white blood cells and fetal trophoblasts, fetal extravillous trophoblasts, fetal endovascular trophoblasts and fetal membrane cells. In some embodiments, of the disclosure, fetal membrane cells are enriched and isolated by methods of the disclosure. In a specific embodiment of the disclosure, the fetal membrane cell is a chorion-and/or an amnion cell. In a further embodiment of the disclosure, the maternal blood sample is from a woman between the gestational ages 15 and 20 weeks.

### Fetal Membrane Markers

Since an object of the disclosure is to isolate fetal membrane cells, ligands directed against markers of fetal membrane cells are used to select fetal membrane cells from other cells present in a blood sample. Preferably, the marker of fetal membrane cells is a marker of chorion and amnion cells. In one embodiment of the disclosure, the fetal membrane cell marker is selected from IGF2, MUC16, UPK1B, EMP1, GPX8, FLT1/VEGFR1, RXFP1, CNR1, PRLR and THY1 and combinations thereof. In one embodiment the combination includes 3 or more, or four or more or five or more, such as six or more, for example seven or more, such as eight or more, for example 9 or more markers.

In another embodiment, the fetal membrane cell marker is selected from MUC16, UPK1B, EMP1, GPX8, FLT1/VEGFR1, RXFP1, CNR1, PRLR, NPR3/NPR-C, FERMT2 and THY1 and combinations thereof. In one embodiment the combination includes 3 or more, or four or more or five or more, such as six or more, for example seven or more, such as eight or more, for example 9 or more markers.

In a third embodiment, the fetal membrane cell marker is selected from IGF2, MUC16, UPK1B, EMP1, GPX8, FLT1/VEGFR1, RXFP1, CNR1, PRLR, NPR3/NPR-C, FERMT2 and THY1 and combinations thereof. In one embodiment the combination includes 3 or more, or four or more or five or more, such as six or more, for example seven or more, such as eight or more, for example 9 or more markers.

In some embodiments, the fetal membrane cell markers are labelled with antibodies against the markers, preferably wherein all antibodies against fetal cell markers are conjugated directly or indirectly to the same fluorophore. Increasing the number of different fetal membrane cell markers labelled increases the likelihood of identifying a fetal membrane cell. In preferred embodiments groups of markers as described are labelled. In other embodiments 3 or more, or four or more or five or more, such as six or more, for example seven or more, such as eight or more, for example 9 or more markers are labelled at the same time.

In one embodiment of the disclosure, the fetal membrane cell marker is for enriching in the optional step of enriching the maternal blood sample for fetal cells. It is preferred that a marker used for enrichment is an extracellular marker, thus in one embodiment of the disclosure, the fetal membrane cell marker for enrichment is one or more of IGF2, MUC16, UPK1B, EMP1, GPX8, FLT1/VEGFR1, RXFP1, CNR1, PRLR and THY1. Accession numbers for the markers can be found in table 1.

TABLE 1

| | Accession Nr. | |
|---|---|---|
| Marker | from NCBI | neXtProt |
| IGF2 | NM_000612 | NX_P01344 |
| MUC16 | NM_024690 | NX_Q8WXI7 |
| UPK1B | NM_006952 | NX_O75841 |
| EMP1 | NM_001423 | NX_P54849 |
| GPX8 | NM_001008397 | NX_Q8TED1 |
| FLT1/VEGFR1 | NM_001159920 | NX_P17948 |
| RXFP1 | NM_001253727 | NX_Q9HBX9 |
| CNR1 | NM_001160226 | NX_P21554 |
| PRLR | NM_000949 | NX_P16471 |
| THY1 | NM_001311160 | NX_P04216 |

In another embodiment, the fetal membrane cell marker for enrichment is selected from MUC16, UPK1B, EMP1, GPX8, FLT1/VEGFR1, RXFP1, CNR1, PRLR, NPR3/NPR-C, FERMT2 and THY1 and combinations thereof (table 1b).

TABLE 1b

Marker
MUC16
NPR3/NPR-C
UPK1B
EMP1
GPX8
CNR1
PRLR
RXFP1 (Relaxin R1)
FLT1/VEGFR1
THY1
FERMT2

In a third embodiment, the fetal membrane cell marker for enrichment is selected from IGF2, MUC16, UPK1B, EMP1, GPX8, FLT1/VEGFR1, RXFP1, CNR1, PRLR, NPR3/NPR-C, FERMT2 and THY1 and combinations thereof.

In some embodiments of the disclosure, the fetal membrane markers are used to enrich and isolate fetal cells.

### Enrichment

The purpose of enrichment is to reduce the number of cells, in particular to reduce the number of maternal cells in the sample. Enrichment can for example be done by MACS or FACS.

### MACS Enrichment

Magnetic-activated cell sorting (MACS) is a method for separation of various cell populations depending on their surface antigens (CD molecules).

Miltenyi Biotec's MACS system uses superparamagnetic nanoparticles and columns. The superparamagnetic nanoparticles are of the order of 100 nm. They are used to tag the targeted cells in order to capture them inside the column. The column is placed between permanent magnets so that when the magnetic particle-cell complex passes through it, the tagged cells can be captured. The column consists of steel wool which increases the magnetic field gradient to maximize separation efficiency when the column is placed between the permanent magnets.

Magnetic-activated cell sorting is a commonly used method often in combination with microbeads which are magnetic nanoparticles conjugated to antibodies which can be used to target specific cells. Magnetic-activated cell sorting can be used to enrich a cell population for desired cells. The magnetic nanoparticles can be conjugated directly to a labelling agent. The magnetic nanoparticles can also be conjugated to another agent capable of binding the labelling agent (secondary labelling).

If MACS is chosen as the enrichment step it is preferred that this step is preceded by a step of contacting cells comprised in the cellular fraction with at least one magnetic ligand directed against a fetal cell marker.

In one embodiment of the disclosure, the optional step of enrichment is conducted prior to contacting the maternal blood sample with a ligand directed against a fetal membrane cell marker and wherein the optional step of enrichment is either negative or positive enrichment depletes the maternal blood sample for maternal cells and/or enriches the maternal blood sample for fetal cells.

In one embodiment of the disclosure, the maternal blood sample is enriched for fetal cells prior to step b.i. In one embodiment of the disclosure, enrichment is done using magnetic activated cell sorting (MACS) or sorting on a fluorescence activated cell sorter (FACS).

In one embodiment of the disclosure, the ligand against maternal blood cells are used for discriminating between fetal cells and maternal blood cells, such as for depleting the maternal blood sample of maternal cells.

### FACS Enrichment

Cell sorting is a method used to purify cell populations based on the presence or absence of specific physical characteristics. In flow cytometers with sorting capabilities, the instrument detect cells using parameters including cell size, morphology, and protein expression, and then droplet technology to sort cells and recover the subsets for post-experimental use.

Flow cytometry cell sorters have a collection system unlike flow cytometry analyzers. The collection process starts when a sample is injected into a stream of sheath fluid that passes through the flow cell and laser intercepts. The stream then carries the cell through a vibrating nozzle. The disturbance in the stream causes it to break into a droplet containing ideally one cell. An electrical charging ring is placed just at the point where the stream breaks into droplets. A charge is placed on the ring based immediately prior to fluorescence intensity being measured, and the opposite charge is trapped on the droplet as it breaks from the stream. The charged droplets then fall through an electrostatic deflection system that diverts droplets into containers based upon their charge. In some systems, the charge is applied directly to the stream, and the droplet breaking off retains charge of the same sign as the stream. The stream is then returned to neutral after the droplet breaks off.

If collected under sterile conditions, the sorted cells can be further cultured, manipulated, and studied.

The sorting on such a FACS can be both single cell sorting and bulk sorting, cells that are discarded and not displaying the given thresholds are discarded.

Gates are set to define how the FACS instrument shall sort cells. Cells that fall within the settings are sorted and cells that fall outside the gates are either sorted for later investigations or discarded. Depending on how the FACS instrument is setup by the user, the sorting can be either a bulk sorting or a single cell sorting. A bulk sorting results in cells falling within the given gate settings to be collected in the same tube. Single cell sorting results in cells falling within the given settings to be individually sorted into collection tubes. The single cell sorting is often more time consuming than a bulk sorting.

As a FACS instrument is capable of sorting several 1000 cells/second, the different setups can vary much depending on the experiment. When sorting into single cell compartments one needs to have a low output, such as a 96 well plate. On the contrary when using bulk sorting, one can sort several thousand cells/tube.

In an embodiment of the disclosure, a bulk sorting is used to enrich a sample. In another embodiment, single cell sorting is used to single cell sort cells, such as for example into single cell compartments.

Depending on the gating strategy of an experiment, one can reduce the amount of agents that needs to be applied to the sample as well as the time used for an experiment, by using a bulk sorting at first followed by a single sorting. Such parameters suitably may include quantifying forward scatter, side scatter and fluorescence emitted from fluorophores excited by lasers within the flow cytometer. Forward scatter is understood as the disturbance in the direction of the light path the object causes and typically correlates with the size of the cell.

Side scatter is understood as the light reflected away from the direction of the light path and typically correlates with the amount of granules in the way of the light path. Emitted fluorescence is quantified when a light source such as a laser excites fluorophores present on the sample and passes through filters known to the person skilled in the art to fit with given lasers and fluorophores. Given a fluorophore with a specific excitation wavelength and emission wavelength, the skilled person is able to select a light source with suitable wavelength, and suitable excitation and emission filters.

When the parameters have been measured by the flow cytometer the cells may be sorted. Suitably this is done by sorting each cell into a separate compartment. The sorted cells are preferably cells that express at least one fetal membrane cell marker and do not express at least one cell marker specific for blood cells. The Gate is chosen to maximize the likelihood of sorting fetal cells while keeping the number of sorted cells relatively low. The remaining—non-sorted—cells are discarded because they are not expected to contain any fetal cells.

In another preferred embodiment, the FACS is used for enrichment, where preferably the sorted cells are collected in a tube before the, now enriched, sample is single cell sorted on the FACS.

In one embodiment, the sample is enriched on a FACS based on the signal from a fetal membrane cell marker.

Thus, If FACS is chosen as the enrichment step it is preferred that this step is preceded by a step of contacting cells comprised in the cellular fraction with at least one fluorescent labelled agent directed against a fetal membrane cell marker.

### FACS Single Cell Sorting

Following the enrichment step, the single cell sorting by FACS can be performed. In a preferred embodiment, cells positive for at least one fetal membrane cell marker and being negative or low for at least one blood cell marker are sorted into single cell compartments. In another preferred embodiment, the signal from a nucleus marker is also included. In a preferred embodiment, the sample is sorted by using the same gating strategy for the enrichment and the single cell sorting. In another embodiment, the sample is sorted by using the same gating strategy for the enrichment and the single cell sorting, with a difference in the size of the gate for the enrichment and the single cell sorting.

### Staining and Microscope

When the method of the disclosure comprises a step of identification, one embodiment comprises detecting the presence of the ligand on or in the fetal cells. Detection and identification can be done by various methods known for the person of skill in the art. In specific embodiments, a microscopic setup or a FACS is used. In a preferred embodiment, FACS is used as described above. In one embodiment of the disclosure, the step of isolating the fetal membrane cell is done using a FACS, such as for example single cell sorting on a FACS or the step is done using picking from a microscope slide, such as either manual or automated picking.

Detection may be enabled by labelling the ligand with fluorescent dyes or other dyes suitable for detection. Thus, the method may e.g. be fluorescent in-situ hybridization (FISH). The probe may comprise a quencher as well as a fluorophore or a FRET pair as described above, which enables detection of hybridisation probes bound to their target sequences. Alternatively or additionally, probes binding to their targets are separated from non-binding probes by one or more washing steps.

Identification may also be done using immunostaining using a ligand such as an antibody. In one embodiment of the disclosure, the method comprises a step of enriching fetal cells from the maternal blood sample and a step of staining fetal membrane cells. In one embodiment of the disclosure, the step of enrichment is followed by a step of staining. In one embodiment of the disclosure, the fetal membrane cell marker is used for staining a fetal membrane cell. In one embodiment of the disclosure, the fetal membrane cell marker for staining a fetal membrane cell is GPX8. In one embodiment of the disclosure, the fetal membrane cell marker for staining a fetal membrane cell is vimentin. In another embodiment of the disclosure, the fetal membrane cell marker for staining a fetal membrane cell is a cytokeratin, such as a cytokeratin selected from table 2, such as CK5, CK17, CK18, CK7, CK8 or CK19. The skilled person know ligands that target multiple cytokeratins, such as a pan-cytokeratin antibody, thus, in one embodiment the ligand targeting fetal membrane cell marker for staining a fetal membrane cell is a pan-cytokeratin antibody.

TABLE 2

| Marker | Accession Nr. |
|---|---|
| CK5 | NM_000424 |
| CK17 | NM_000422 |
| CK18 | NM_000224 |
| CK7 | NM_005556 |
| CK8 | NM_001256293 |
| CK19 | NM_002276 |
| VIMENTIN | NM_003380 |

In an embodiment of the disclosure, the fetal membrane cell marker for staining a fetal membrane cell is a cytokeratin, such as a cytokeratin selected from CK5, CK17, CK18, CK7, CK8, CK19, Vimentin and combinations thereof.

In another embodiment of the disclosure, the fetal membrane cell marker for staining a fetal membrane cell is CK5, CK7, CK8, CK17, CK18, CK19, Ubiquitin, Pan CK or Vimentin and combinations thereof (table 2b).

TABLE 2b

Marker
CK5
CK7
CK8
CK17
CK18
CK19
Pan CK
Vimentin
Ubiquitin

In one embodiment of the disclosure, the Pan CK antibody targets the cytokeratins CK1, CK2, CK4, CK5, CK6, CK7, CK8, CK71, CK72, CK75 or CK78.

One type of a microscopic setup is a microscope equipped with a cell picker. A microscope can also be setup with a fluorescence scanner. Typically, a sample positively enriched for fetal cells and stained using fetal cell specific markers conjugated to a fluorescent dye, or bound with fluorescent secondary antibodies, is smeared on a glass slide, or a slide containing microwells. Glass slides or slides containing microwells are scanned on a fluorescence microscope which has a software to identify fetal cells based on the fluorescent signals that are emitted from the dye. The microscope can have an integrated cell picker to pick the cells off the glass slides or microwells either manually or automatically. The microscope is integrated with a fluorescence scanner that can scan either glass slides or slides containing microwells. The scanner can have a software designed to identify fetal cells based on fluorescent signals emitted from the dye conjugated to the fetal cell specific markers. Positively identified fetal cells can be picked off the glass slide or slides containing microwells using the cell picker integrated with the scanner.

### Maternal Markers

Ligands that bind specifically to maternal cells may also in one embodiment be used for enrichment. Thus, in an embodiment, the method further comprises a step of contacting the sample with a maternal cell specific ligand directed to a maternal antigen. This step may be performed at any time suitable. After contacting the sample with a maternal cell specific ligand, enrichment may e.g. be done using as described above.

Preferably, the ligand is selected from the group consisting of ligands that bind to antigens encoded by mRNAs preferentially expressed in maternal blood cells but not in fetal cells as identified by the present inventors.

As will be clear to the person skilled in the art, additional antigen dependent enrichment steps (negative selections) based on ligands (or antigens) known from the prior art may be used. Thus, in one embodiment, an additional antigen dependent enrichment step is performed, where the ligand is selected from the group consisting of ligands that bind to maternal specific antigens known from the prior art such as CD45, HLA-A, HLA-B or antibodies selected from the group consisting of HLe-1, M3 and L4. In further embodiments, the method comprises a step of contacting the maternal blood sample with a ligand against CD45, CD3, CD14, CD15, CD16, and/or CD19. Preferably a combination of CD45 and CD14 is used.

A preferred cell type marker for negative selection is CD45 also known as leukocyte common antigen. CD45 is a transmembrane protein expressed by all differentiated hematopoietic cells except erythrocytes and plasma cells. The CD45 protein exists in different forms which are all produced from a single complex gene giving rise to eight different mature mRNAs and resulting in eight different protein products. It is expressed on all leukocytes but not on other cells, and thus functions as a pan-leukocyte marker including the different and diverse types of leukocytes (or white blood cells) such as neutrophils, eosinophils, basophils, lymphocyte (B and T cells), monocytes and macrophages.

Due to the expression of CD45 on a large majority of the nucleated cells present in maternal blood a negative selection using the CD45 marker is preferred. Following depletion of CD45 positive cells, the CD45 negative cells of the sample is collected. Such depletion and collection can be performed by any suitable method known in the art.

In one embodiment the cells present in the maternal blood sample or a fragment thereof is counterstained using a CD45 marker at any suitable time point thereby identifying the maternal cells present in the sample. The CD45 negative cells of the sample may then be collected, or sorted out when performing FACS for positive isolation of fetal cells. Such a counterstain and collection may be performed using any suitable method known in the art. CD45 is preferably labelled in combination with CD14.

### Nuclear Dyes

To differentiate nucleated cells from debris, a marker for a eukaryotic cell is preferably used. For a person skilled in the art, such a dye will typically be a DNA-intercalating dye and can be dyes such as any Hoechst dye, DAPI, propidium iodide, 7-AAD, Vybrant DyeCycle Stains, SYTOX stains, or SYTO stains. In another embodiment the dye is Vybrant DyeCycle Ruby Stain.

In one embodiment of the disclosure, the method further comprises a step of contacting the maternal blood sample with a fluorescent labelling agent directed against the nucleus, such as a dye described above. In one embodiment of the disclosure, the fluorescent labelling agent directed against the nucleus is used for identifying cells in the maternal blood sample.

### Ligands

A particular feature of the disclosure is the specific labelling of cells using ligands. These ligands can be any type of molecule that is capable of binding specifically to a particular type of cell, e.g. through binding to a marker on the surface or inside the cells. In one embodiment of the disclosure, the ligand directed against a fetal membrane cell marker is a magnetic-and/or a fluorescent ligand.

A ligand may, according to the disclosure, be an antibody, such as any suitable antibody known in the art including other immunologically active fragments of antibodies or single chain antibodies. Antibody molecules are typically Y-shaped molecules whose basic unit consist of four polypeptides, two identical heavy chains and two identical light chains, which are covalently linked together by disulfide bonds. Each of these chains is folded in discrete domains. The C-terminal regions of both heavy and light chains are conserved in sequence and are called the constant regions, also known as C-domains. The N-terminal regions, also known as V-domains, are variable in sequence and are responsible for the antibody specificity. The antibody specifically recognizes and binds to an antigen mainly through six short complementarity-determining regions located in their V-domains.

The ligand may be a single moiety, e.g., a polypeptide or protein, or it may include two or more moieties, e.g., a pair of polypeptides such as a pair of single chain antibody domains. Methods of generating antibodies are well known to a person skilled in the art, by immunisation strategies for the generation of monoclonal or polyclonal antibodies or in vitro methods for generating alternative binding members. Polyclonal antibodies may be such as sheep, goat, rabbit or rat polyclonal antibody.

In addition any suitable molecule capable of high affinity binding may be used including antibody fragments such as single chain antibodies (scFv), particularly, Fab and scFv antibodies which may be obtained by phage-display or single domain antibodies (VHH) or chimeric antibodies. The ligand may be derived from a naturally occurring protein or polypeptide; it may be designed de novo, or it may be selected from a library. For example, the ligand may be derived from an antibody, a single chain antibody (scFv), a single domain antibody (VHH), a lipocalin, a single chain MHC molecule, an Anticalin™ (Pieris), an Affibody™, a nanobody (Ablynx) or a Trinectin™ (Phylos). Thus, methods of generating binding members of various types are well known in the art.

In one embodiment of the disclosure, the ligand is a fragment of an antibody, preferably an antigen binding fragment or a variable region. Examples of antibody fragments useful with the present disclosure include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′).

Additional fragments can include diabodies, linear antibodies, single-chain antibody molecules, and multispecific antibodies formed from antibody fragments.

In one preferred embodiment, the present disclosure relates to ligands derived from a naturally occurring protein or polypeptide; said protein or polypeptide may for example be designed de novo, or may be selected from a library. The ligand may be a single moiety, e.g., a polypeptide or protein domain, or it may include two or more moieties, e.g., a pair of polypeptides. The ligand may for example, but exclusively, be a lipocalin, a single chain MHC molecule, an Anticalin™ (Pieris), an Affibody™ (Affibody), or a Trinectin™ (Phylos), Nanobodies (Ablynx). The ligand may be selected or designed by recombinant methods known by people well known in the art.

In another embodiment of the disclosure, the ligand is an affibody, such as any suitable affibody known in the art, in particular antibodies as defined herein, such as affibodies or immunological fragments of affibodies. Affibodies are selected in vitro, from an affibody library constructed by combinatorial variation of the IgG binding domain of Protein A. Protein A is a surface protein from the bacterium Staphylococcus aureus. The binding domain consists of 58 residues, where of 13 are randomized to generate Affibody® libraries Thus, the size of an affibody is considerably less than of an antibody (www.affibody.com).

Finally, the ligand may be a receptor ligand directly or indirectly linked to a fluorophore. Accordingly, it is an object of the present disclosure to provide for the selective labelling of fetal cells in the maternal biological sample based on a hybridisation technique. Alternatively, probes recognising mRNA selectively expressed by fetal cells may be used.

Fetal cell specific RNA, generally messenger RNA (mRNA) sequences may be used as fetal cell markers. The presence of such mRNA indicates that the gene for the fetal protein is being transcribed and expressed. The probes used to label fetal cells in a sample containing fetal and maternal cells include nucleic acid molecules, which comprise the nucleotide sequence complementary to the nucleotide sequence of the RNA molecule encoding a specific protein. Fetal cells contain distinct mRNAs or RNA species that do not occur in other cell types. The detection of these RNAs, can serve to label cells of fetal origin.

Likewise, maternal cells may be labelled using a probe specific for a maternal cell mRNA. Thus, in some embodiments, the ligand is a probe, such as a nucleic acid probe as described herein.

According to the disclosure the probe may be any type of probe known in the art for detection of RNA or DNA molecules. Conventional probes known by a person skilled in the art comprise, RNA and DNA probes synthesised from nucleotides of deoxynucleotide, respectively using a commercial synthesiser. Probes may be comprised of the natural nucleotide bases or known analogues of the natural nucleotide bases. It is further contemplated that the probes may be oligomers comprising one or more nucleotide analogues including peptide nucleic acids and other synthetic molecules capable of Watson Crick-base pairing.

For detection of chromosomal DNA, Fluorescence In Situ Hybridization (FISH) is frequently employed.

In one embodiment, a ligand (such as an antibody) or a synthetic probe is directly labelled, by having fluorophores covalently attached thereto. In another embodiment, a ligand (such as an antibody) or a synthetic probe is indirectly labelled, by being bound by a second agent having fluorophores covalently attached thereto. The binding of such probes or ligands to the target in the cell may be observed under a microscope as a bright fluorescence or may be detected by a FACS.

By use of a combination of labelling methods it is possible to enhance the signals from the fetal cells, thereby facilitating the identification thereof.

In order to enhance the probability and/or selectivity of identifying the fetal cells over the background of maternal cells, two or more selective ligands may be performed. The two or more ligands may be a combination of any of the ligands used for single labelling described above. Accordingly, the combined labelling may be carried out by the use of two or more different hybridisation probes, such as a combination of a DNA probe and a PNA or LNA probe for hybridisation with the same fetal RNA or more preferred with different fetal RNAs. Also, two or more different DNA probes (or PNA probes, or similar probes capable of specific hybridisation) may be used for hybridisation with different fetal RNAs. Likewise a combination of different ligands may be used, either with specificity for the same fetal antigen or with specificity for different antigens. In further embodiments labelling with a combination of nucleotide probes and ligands may be performed.

The fluorophore is selected to emit in the wave-length area of the detection means, and furthermore in suitable combination with an optional second labelling. In particular the fluorophores may be selected from FITC (fluorescein-isofluocyanate) or TRITC (Rhodanine Tetramethyl-isofluocyanate) having excitation at 495 nm and 520-530 nm, respectively. Further fluorophores which may be used are listed in the following tables with wavelength of excitation and emission of various fluorochromes:

TABLE 3
Reactive and conjugated probes

| Fluorochome | Ex (nm) | Em (nm) | MW | Notes |
|---|---|---|---|---|
| Hydroxycoumarin | 325 | 386 | 331 | Succinimidyl ester |
| Aminocoumarin | 350 | 445 | 330 | Succinimidyl ester |
| Methoxycoumarin | 360 | 410 | 317 | Succinimidyl ester |
| Cascade Blue | 375; 400 | 423 | 596 | Hydrazide |
| Lucifer yellow | 425 | 528 | | |
| NBD | 466 | 539 | 294 | NBD-X |
| R-Phycoerythrin (PE) | 480; 565 | 578 | 240 | |
| PE-Cy5 conjugates | 480; 565; 650 | 670 | | aka Cychrome, |
| | | | | R670, Tri-Color, |
| | | | | Quantum Red |
| PE-Cy7 conjugates | 480; 565; 743 | 767 | | |
| APC-Cy7 conjugates | 650; 755 | 767 | | PharRed |
| Red 613 | 480; 565 | 613 | | PE-Texas Red |
| Fluorescein | 495 | 519 | 389 | FITC; pH sensitive |
| FluorX | 494 | 520 | 587 | (AP Biotech) |
| BODIPY-FL | 503 | 512 | | |
| Tetramethylrhodamine | 550 | 560-608 | 444 | TRITC |
| Tetramethylrhodamine | 547 | 530-560 | | |
| isothiocyanate | | | | |
| X-Rhodamine | 570 | 576 | 548 | XRITC |
| Lissamine Rhodamine B | 570 | 590 | | |
| PerCP | 490 | 675 | | Peridinin chlorphyll |
| | | | | protein |
| Texas Red | 589 | (603) 615 | 625 | Sulfonyl chloride |
| Allophycocyanin (APC) | 650 | 660 | 104 | |
| TruRed | 490, 675 | 695 | | PerCP-Cy5.5 conjugate |

TABLE 4
Alexa Fluor dyes (Molecular Probes)

| Fluorochome | Ex (nm) | Em (nm) | MW |
|---|---|---|---|
| Alexa Fluor 350 | 346 | 445 | 410 |
| Alexa Fluor 430 | 430 | 545 | 701 |
| Alexa Fluor 488 | 494 | 517 | 643 |
| Alexa Fluor 532 | 530 | 555 | 724 |
| Alexa Fluor 546 | 556 | 573 | 1079 |
| Alexa Fluor 555 | 556 | 573 | 1250 |
| Alexa Fluor 568 | 578 | 603 | 792 |
| Alexa Fluor 594 | 590 | 617 | 820 |
| Alexa Fluor 633 | 621 | 639 | 1200 |
| Alexa Fluor 647 | 650 | 668 | 1250 |
| Alexa Fluor 660 | 663 | 690 | 1100 |
| Alexa Fluor 680 | 679 | 702 | 1150 |
| Alexa Fluor 700 | 696 | 719 | |
| Alexa Fluor 750 | 752 | 779 | |

TABLE 5
Spectrum dyes (Vysis)

| Fluorochome | Ex (nm) | Em (nm) |
|---|---|---|
| SpectrumOrange | 559 | 588 |
| SpectrumGreen1 | 497 | 524 |
| SpectrumGreen2 | 509 | 538 |
| SpectrumAqua | 433 | 480 |
| SpectrumBlue | 400 | 450 |
| SpectrumGold | 530 | 555 |
| SpectrumRed | 592 | 612 |
| SpectrumFRed (far red) | 655 | 675 |

TABLE 6
Cy Dyes (AP Biotech)

| Fluorochome | Ex (nm) | Em (nm) | MW |
|---|---|---|---|
| Cy2 | 489 | 506 | 714 |
| Cy3 | (512); 550 | 570; (615) | 767 |
| Cy3.5 | 581 | 596; (640) | 1102 |
| Cy5 | (625); 650 | 670 | 792 |
| Cy5.5 | 675 | 694 | 1128 |
| Cy7 | 743 | 767 | 818 |

Unlabelled ligands may be used as known in the art, by use of a second labelling step with e.g. a secondary antibody against the unlabelled primary antibody, said antibody being labelled as discussed above, such as fluorophore labelled. By this two-step it may be possible to enhance the signals from the fetal cells. Further detection steps may be included by using indirect labelling as described here below.

Fluorescently labelled antibodies can be labelled directly or indirectly by at least one fluorophore. A person skilled in the art, will be able to select suitable fluorophores and can be any fluorophore such as Alexa Fluor 488, Alexa Fluor 555, Fluorescein isothiocyanate (FITC), Phycoerythrin (PE), BV421.

When more than one fetal membrane cell marker are used, these markers may be linked to the same fluorophore. This is because often there is no need to discriminate between cells having the different markers. The same applies to cases where more than one maternal cell marker is used.

Indirect labelling of fluorescently labelled ligands can be done by attaching a fluorophore to secondary antibodies binding the ligand bound to the markers.

In a preferred embodiment, the secondary antibody may be selected from FITC (fluorescein-isofluocyanate) or TRITC (Rhodanine Tetramethyl-isofluocyanate) having excitation at 495 nm and 520-530 nm, respectively. Further fluorophores which may be used are listed in the above table. In another preferred embodiment, the secondary antibody may be selected from Alexa Fluor 488 or Alexa Fluor 555 having excitation at 490 nm and 555 nm, respectively.

In one embodiment of the disclosure, the fluorescent ligand is directly labelled by at least one fluorophore. In one embodiment of the disclosure, the fluorescent ligand is indirectly labelled by at least one fluorophore. In a preferred embodiment, the fluorophore is selected from the group consisting of Alexa Fluor 488, Alexa Fluor 555, Fluorescein isothiocyanate (FITC), Phycoerythrin (PE), and BV421.

In one embodiment of the disclosure, the ligand directed against a fetal membrane cell marker is a ligand selected from antibodies, nucleotide probes, receptor ligands, and other specific binding molecules.

### Diagnosing

It is an object of the disclosure to provide improved methods for diagnosing fetuses, through the methods disclosed herein. Preferably, the diagnosis can be done without harming the fetus. Noninvasive prenatal testing (NIPT), sometimes called noninvasive prenatal screening (NIPS), is a method of determining the risk that the fetus will be born with certain genetic abnormalities. NIPT can be used to look for chromosomal disorders that are caused by the presence of an extra or missing copy (aneuploidy) of a chromosome. NIPT may include screening for additional chromosomal disorders that are caused by missing (deleted) or copied (duplicated) sections of a chromosome. NIPT can also be used to test for genetic disorders that are caused by changes (variants) in single genes. NIPT is considered noninvasive because it requires drawing blood only from the pregnant woman and does not pose any risk to the fetus.

Thus, it is an object of the disclosure to detect genetic abnormalities in the genome of the fetal cell.

In one embodiment of the disclosure, the diagnosis is done by analysing the genotype or the phenotype of the fetal membrane cell. In one embodiment of the disclosure, the genotype is obtained by STR analysis or SNP analysis. In one embodiment of the disclosure, the phenotype is diagnosed by detecting one or more markers associated with a genetic abnormality in the genome of the fetal cell. In one embodiment of the disclosure, the genetic abnormality is aneuploidy, monosomy, polysomy, trisomy, copy number variation (CNV), single nucleotide variation (SNV), or a monogenic disorder.

In one embodiment of the disclosure, the genetic abnormality is detected by one or more methods selected from Microarray-based Comparative Genomic Hybridization (aCGH), Short Tandem Repeat analysis (STR analysis), whole genome amplification, whole genome scan, SNP array, Polony sequencing, Shotgun sequencing, Massively parallel signature sequencing (MPSS), Sanger Sequencing, PCR-based methods and Next-Generation Sequencing methods such as Illumina (Solexa) sequencing, Roche 454 sequencing, Ion torrent: Proton/PGM sequencing and/or SOLiD sequencing.

### Sample

In some embodiments of the disclosure, the samples used in the methods of the disclosure are biological samples. In a preferred embodiment, the biological sample is a blood sample, such as a peripheral blood sample. The blood sample can have a volume of 5-30 ml. The samples can be obtained in any tube suitable for blood samples. In another preferred embodiment, the blood sample can have a volume of 2-100 mL, such as 3-70, 5-50 mL, 5-30 mL and 10-30 mL. In even further embodiments, the blood sample is between 5-50 mL, such as 5 mL, 10 mL, 15 mL, 20 mL, 25 mL, 30, mL or 40 mL.

In one embodiment, a cellular fraction is separated from the biological sample, suitably by centrifuging said biological sample. Centrifugation of the biological sample will preferably be between 0 and 25 minutes, more preferably between 0 and 15 minutes and most preferably between 5 and 10 minutes. The centrifugal force applied to the sample will preferably be between 0 and 5000 G, more preferably between 500 and 3000 G and most preferably between 1000 and 2000 G, such as 1600 G.

A person skilled in the art will know the centrifugation to separate a blood sample into a plasma-and cellular fraction.

In one embodiment of the disclosure, a cellular fraction is separated from plasma of said blood sample. In one embodiment of the disclosure, said cellular fraction is separated from said plasma fraction by centrifugation. In one embodiment of the disclosure, said cellular fraction comprises both maternal cells and fetal cells. In one embodiment of the disclosure, said cellular fraction comprises red blood cells, white blood cells and fetal trophoblasts, fetal extravillous trophoblasts, fetal endovascular trophoblasts and fetal membrane cells.

Cervical Smear

In another embodiment, the biological sample is a cervical smear. Mucus from the swab may first be dissolved using acetic acid or DDT. The cellular fraction achieved after this step may then be fixed in paraformaldehyde. The cellular fraction from the cervical smear comprises fetal cells (trophoblasts, such as cytotrophoblasts, syncytiotrophoblast and/or interstitial trophoblasts) and squamous epithelial cells, columnar epithelial cells, white blood cells and red blood cells. The cellular fraction also comprises amnion and chorion cells. Following that, the fetal cells may be enriched and stained as herein described for blood samples.

### Fixation of the Cells of a Maternal Sample

In one embodiment of the disclosure, fixation of the cells of the sample greatly increases stability of fetal cells in the sample, while allowing enrichment and identification of fetal cells e.g. as further described herein above. In one embodiment the fixation procedure can be performed on a non-enriched sample immediately after sampling, resulting in fixation of cellular components in the maternal sample. At the same time the fixation is so mild that maternal erythrocytes can be lysed selectively in a subsequent lysis step.

Fixation is preferably done for between 1 and 60 minutes. More preferably fixation is done for between 5 and 30 minutes and most preferably, fixation is done between 5 and 15 minutes such as 10 minutes.

The fixation solution preferably comprises between 0.5% and 7.5% paraformaldehyde, more preferably 1% and 6% and most preferably between 1.5% and 2%.

In addition to paraformaldehyde, the fixation solution preferably comprises salt at a concentration between 0.05 M and 0.3 M. More preferably the salt concentration is between 0.1 M and 0.2 M and most preferred is a concentration between 0.125 M and 0.175 M.

In one embodiment of the disclosure, the method further comprising a step of fixating the blood cells subsequent to being separated from said plasma fraction, wherein fixation is preferably a paraformaldehyde fixation.

### Lysing Step

Preferably, the fixation step may be followed by a step of lysis of red blood cells comprising contacting the fixated sample with a lysis buffer.

The lysis buffer typically comprises a non-ionic detergent, preferably Triton-X-100. Preferred concentrations of the detergent are between 0.01% (w/w) and 0.5%, more preferably between 0.05%-0.3%, and most preferably 0.1%.

In a preferred embodiment, the lysis step is performed immediately after the fixation step. I.e. the lysis solution is added directly to the sample, e.g. after fixation for 10 minutes. Lysis is typically done for a period of 1 minutes to 120 minutes, more preferably 5 to 60 minutes and most preferably for 6 to 10 minutes.

The lysis buffer can, in addition to lysis of red blood cells, also create small openings in the cell membranes of the white blood cells which allows the labelling agents to penetrate the cell membrane and bind to their target antigens. In one embodiment of the disclosure, the method further comprising a step of selectively lysing red blood cells of said cellular fraction using a detergent subsequent to separation of said cellular fraction from said plasma fraction, wherein said lysing also permeabilizes the remaining cells in said cellular fraction.

### Specific Embodiments

In a further aspect, the disclosure relates to a method of detecting a fetal cell comprising the steps of:
a) providing a maternal blood sample obtained from a woman carrying a fetus;
b) contacting the cells comprised in the maternal blood sample with a ligand towards one or more fetal membrane cell markers selected from IGF2, MUC16, UPK1B, EMP1, GPX8, FLT1/VEGFR1, RXFP1, CNR1, PRLR and THY1; and
c) detecting the fetal cell marker, thereby detecting the fetal cell.

In an event further aspect, the disclosure relates to a method of prenatal diagnosis comprising the steps of:
a) providing a maternal blood sample obtained from a woman carrying a fetus;
b) contacting the cells comprised in the maternal blood sample with a ligand towards one or more fetal membrane cell markers selected from IGF2, MUC16, UPK1B, EMP1, GPX8, FLT1/VEGFR1, RXFP1, CNR1, PRLR and THY1;
c) detecting the fetal cell marker;
d) isolating the fetal cell; and
e) diagnosing the fetal cell.

In a further aspect, the disclosure relates to a method of detecting a fetal cell comprising the steps of:
a) providing a maternal blood sample obtained from a woman carrying a fetus;
b) contacting the cells comprised in the maternal blood sample with a ligand towards one or more fetal membrane cell markers selected from MUC16, UPK1B, EMP1, GPX8, FLT1/VEGFR1, RXFP1, CNR1, PRLR, THY1, NPR3/NPR-C and FERMT2; and
c) detecting the fetal cell marker, thereby detecting the fetal cell.

In an event further aspect, the disclosure relates to a method of prenatal diagnosis comprising the steps of:
a) providing a maternal blood sample obtained from a woman carrying a fetus;
b) contacting the cells comprised in the maternal blood sample with a ligand towards a fetal cell marker selected from MUC16, UPK1B, EMP1, GPX8, FLT1/VEGFR1, RXFP1, CNR1, PRLR, THY1, NPR3/NPR-C and FERMT2;
c) detecting the fetal cell marker;
d) isolating the fetal cell; and
e) diagnosing the fetal cell.

In a further aspect, the disclosure relates to a method of detecting a fetal cell comprising the steps of:
a) providing a maternal blood sample obtained from a woman carrying a fetus;
b) contacting the cells comprised in the maternal blood sample with a ligand towards a fetal cell marker selected from IGF2, MUC16, UPK1B, EMP1, GPX8, FLT1/VEGFR1, RXFP1, CNR1, PRLR, THY1, NPR3/NPR-C and FERMT2; and
c) detecting the fetal cell marker, thereby detecting the fetal cell.

In an event further aspect, the disclosure relates to a method of prenatal diagnosis comprising the steps of:
a) providing a maternal blood sample obtained from a woman carrying a fetus;
b) contacting the cells comprised in the maternal blood sample with a ligand towards a fetal cell marker selected from IGF2, MUC16, UPK1B, EMP1, GPX8, FLT1/VEGFR1, RXFP1, CNR1, PRLR, THY1, NPR3/NPR-C and FERMT2;
c) detecting the fetal cell marker;
d) isolating the fetal cell; and
e) diagnosing the fetal cell.

In certain embodiments, the two methods of above also employs other markers as described previously, such as maternal markers and/or fetal cell specific markers, such as cytokeratins and/or Vimentin as discussed above.

### EXAMPLES

### Example 1 Blood Processing and Staining

Blood samples are collected from pregnant women between the gestational ages of 15 to 20 weeks. Samples are processed and fetal cells enriched using the following methods:

### Blood Processing

A total of 30 mL of peripheral blood is collected in Cell-Free DNA BCT® Tubes (Streck, Omaha, USA). The blood samples are centrifuged at 1000-2000 g for 2-10 minutes and plasma is removed. The blood samples are fixed in a 1-10% formaldehyde buffer in phosphate-buffered saline (PBS), followed by lysis of red blood cells in 0.1-1% Triton-X-100 detergent in PBS. The cells are subsequently washed in PBS and a 0.5% bovine serum albumin buffer and pelleted before fetal cell enrichment.

### Fetal Cell Enrichment and Staining Using Miltenyi's Magnetic Activated Cell Sorting (MACS)

Fetal cell enrichment are performed using MACS, (Miltenyi Biotec, Germany). The pelleted unenriched cells after ‘blood processing’ are incubated with antibodies against one or more of the markers shown in table 7 for 15 minutes to 1 hour. The cells are then incubated with secondary antibody conjugated to magnetic beads, and the cell suspension is applied to MACS columns for enrichment. The retained cells are plunged in a 15 mL tube and washed (centrifugation for 10 minutes), before reapplying the enriched cells to a second column for antibody staining.

TABLE 7

| Marker | Antibody |
|---|---|
| IGF2, mouse | |
| MUC16, mouse | abcam ab1107 |
| UPK1B, mouse | sigmaaldrich |
| EMP1, mouse | abnova H00002012- |
| GPX8, mouse | abnova H00493869- |
| FLT1/VEGFR1, mouse | abcam ab212369 |
| RXFP1, mouse | rndsystems mab8898 |
| CNR1, mouse | sigma wh0001268m1 |
| PRLR, mouse | abcam u5 ab2772 |
| PRLR, mouse | abcam t6 ab2773 |

The staining is performed on the column by incubating the enriched cell fraction with antibodies against CK5, CK7, CK8, CK17, CK18, CK19, Pan CK, Vimentin that target fetal cells, and CD14 and CD45 antibodies that target maternal white blood cells, for 15 minutes to 1 hour. After incubation with the primary antibodies, the cells in the columns are rinsed with PBS. Following that, secondary antibodies labelled with fluorescent dyes are applied, and the cells incubated. The stained cells are washed in PBS before being plunged and saved at 4° C. until FACS or manual picking.

### Example 2

The enriched cells of example 1 are stained with a nuclear dye and sorted on a FACS and fetal origin confirmed by STR analysis.

### Example 3

The enriched cells of example 1 are stained with a nuclear dye and smeared onto glass slides and classified using CellCelector (fluorescence scanner—cell-picker) and fetal origin confirmed by STR analysis.

### Example 4

Cells are prepared as described in example 1, with the amendment that the cells are stained with antibodies of table 7. The cells are then enriched on a FACS and investigated further by either resorting on a FACS (as in example 2) or by manual picking (as in example 3). A nuclear stain and antibodies targeting maternal white blood cells are also included. Following isolation, fetal origin is confirmed by STR analysis.

### Example 5

Cells are prepared as described in example 1, 2, 3 and/or 4, with the amendment that antibodies against THY1 is used. A nuclear stain and antibodies targeting maternal white blood cells are also included. Following isolation, fetal origin is confirmed by STR analysis.

### Example 6

A total of 30 mL of peripheral blood is collected in Cell-Free DNA BCT® Tubes (Streck, Omaha, USA), from five pregnant women between the gestational ages of 15 to 20 weeks.

### Blood Processing

Blood samples are processed as described in example 1.

### Fetal Cell Enrichment and Staining Using Miltenyi's Magnetic Activated Cell Sorting (MACS)

Fetal cell enrichment is performed using MACS, (Miltenyi Biotec, Germany). The pelleted unenriched cells after ‘blood processing’ are incubated with antibodies against the markers shown in table 8, for 15 minutes to 1 hour. The cells are then incubated with secondary antibody conjugated to magnetic beads, and the cell suspension is applied to MACS columns for enrichment. The retained cells are plunged in a 15 mL tube and washed (centrifugation for 10 minutes), before reapplying the enriched cells to a second column for antibody staining.

TABLE 8

| Marker | Antibody (company, cat. number) |
|---|---|
| MUC16 | (Abcam, ab697) |
| NPR3/NPR-C | (Novus, NBP2-02401) |
| UPK1B | (Abcam, ab263454) |
| EMP1 | (Abnova, H00002012-A01) |
| GPX8 | (Abnova, H00493869-B01P) |
| CNR1 | (Novus H00001268-M01) |
| PRLR (t6) | (Abcam, ab2773 (T6)) |
| PRLR (u5) | (Abcam, ab2772 (u5)) |
| RXFP1 (Relaxin R1) | (Novus, H00059350-M01) |
| FLT1/VEGFR1 | (Novus, NBP2-30982) |
| THY1 | (Abcam, ab181469) |
| FERMT2 | (Abcam, ab254535) |

The staining is performed on the column by incubating the enriched cell fraction for 15 minutes to 1 hour with antibodies against CK5, CK7, CK8, CK17, CK18, CK19, Pan CK, Vimentin, Ubiquitin (these antibodies target fetal cells, and are directly conjugated to a fluorescent dye AF488) and CD14 and CD45 antibodies that target maternal white blood cells and are directly conjugated to a fluorescent dye PE (Table 9). The stained cells are washed in PBS before being plunged and saved at 4° C. until FACS.

TABLE 9

| | Antibody (company, | |
|---|---|---|
| Marker | cat. number) | Fluorophore |
| CK5 | (Abcam, ab193894) | AF488 |
| CK7 | (Abcam, ab185048) | AF488 |
| CK8 | (Abcam, ab192467) | AF488 |
| CK17 | (Abcam, ab185032) | AF488 |
| CK18 | (Abcam, ab194124) | AF488 |
| CK19 | (Abcam, ab192643) | AF488 |
| Pan CK | (Bioss, BS-1712R-A488) | AF488 |
| Vimentin | (Abcam, ab185030) | AF488 |
| Ubiquitin | (Novus, NB300-676AF488) | AF488 |
| CD45 | (Bioss, bs-4820R-PE) | PE |
| CD14 | (Bioss, bs-1192R-PE) | PE |

### FACS and STR Analysis

The enriched and stained cells are stained with Hoechst as a nuclei dye. Using FACS, the cells were then single-cell-sorted individually into PCR tubes if they are:
Hoechst positive
Positive for the antibodies coupled to the AF488 fluorophore
Negative for CD45+CD14 which are coupled to the PE fluorophore

The origin (maternal/fetal) of the sorted cells is confirmed by STR analysis.

Five samples are run:

| | Number of fetal |
|---|---|
| | cells confirmed by |
| Sample | STR |
| 3439 | 0 |
| 3440 | 0 |
| 3446 | 0 |
| 3454 | 4 |
| 3455 | 1 |

### Example 7

A total of 60 mL of peripheral blood, amounting to two blood samples (named with suffixes ‘A’ and ‘B’ after the sample ID), is collected in Cell-Free DNA BCT® Tubes (Streck, Omaha, USA), from five pregnant women between the gestational ages of 15 to 20 weeks.

### Blood Processing

Blood samples are processed as described in example 1.

### Fetal Cell Enrichment and Staining Using Miltenyi's Magnetic Activated Cell Sorting (MACS)

Fetal cell enrichment is performed using MACS, (Miltenyi Biotec, Germany). The pelleted unenriched cells in samples ‘A’ after ‘blood processing’ are incubated with antibodies against the markers shown in table 8 for 15 minutes to 1 hour. The pelleted unenriched cells in samples ‘B’ after ‘blood processing’ are incubated with antibodies against the markers shown in table 8 except NPR3/NPR-C, FERMT2, for 15 minutes to 1 hour. Table 10 shows the comparison of antibodies used in samples ‘A’ and ‘B’. The cells are then incubated with secondary antibody conjugated to magnetic beads, and the cell suspension is applied to MACS columns for enrichment. The retained cells are plunged in a 15 mL tube and washed (centrifugation for 10 minutes), before reapplying the enriched cells to a second column for antibody staining.

TABLE 10

| Marker | | Antibody (company, |
|---|---|---|
| ‘A’ samples | ‘B’ samples | cat. number) |
| MUC16 | MUC16 | (Abcam, ab697) |
| NPR3/NPR-C | — | (Novus, NBP2-02401) |
| UPK1B | UPK1B | (Abcam, ab263454) |
| EMP1 | EMP1 | (Abnova, H00002012-A01) |
| GPX8 | GPX8 | (Abnova, H00493869-B01P) |
| CNR1 | CNR1 | (Novus H00001268-M01) |
| PRLR (t6) | PRLR (t6) | (Abcam, ab2773 (t6)) |
| PRLR (u5) | PRLR (u5) | (Abcam, ab2772 (u5) |
| RXFP1 | RXFP1 | (Novus, H00059350-M01) |
| (Relaxin R1) | (Relaxin R1) | |
| FLT1/VEGFR1 | FLT1/VEGFR1 | (Novus, NBP2-30982) |
| THY1 | THY1 | (Abcam, ab181469) |
| FERMT2 | — | (Abcam, ab254535) |

The staining in samples ‘A’ is performed on the column by incubating the enriched cell fraction for 15 minutes to 1 hour with antibodies listed in table 9. The staining in samples ‘B’ is performed on the column by incubating the enriched cell fraction for 15 minutes to 1 hour with antibodies listed in table 9, except Ubiquitin. Table 11 shows the comparison of staining antibodies used in samples ‘A’ and ‘B’. The stained cells are washed in PBS before being plunged and saved at 4° C. until FACS.

TABLE 11

| Marker | | | Antibody (company, |
|---|---|---|---|
| ‘A’ samples | ‘B’ samples | Fluorophore | cat. number) |
| CK5 | CK5 | AF488 | (Abcam, ab193894) |
| CK7 | CK7 | AF488 | (Abcam, ab185048) |
| CK8 | CK8 | AF488 | (Abcam, ab192467) |
| CK17 | CK17 | AF488 | (Abcam, ab185032) |
| CK18 | CK18 | AF488 | (Abcam, ab194124) |
| CK19 | CK19 | AF488 | (Abcam, ab192643) |
| Pan CK | Pan CK | AF488 | (Bioss, BS-1712R-A488) |
| Vimentin | Vimentin | AF488 | (Abcam, ab185030) |
| Ubiquitin | — | AF488 | (Novus, NB300-676AF488) |
| CD45 | CD45 | PE | (Bioss, bs-4820R-PE) |
| CD14 | CD14 | PE | (Bioss, bs-1192R-PE) |

### FACS and STR Analysis

The enriched and stained cells are stained with Hoechst as a nuclei dye. Using FACS, the cells were then single-cell-sorted individually into PCR tubes if they are:
Hoechst positive
Positive for the antibodies coupled to the AF488 fluorophore
Negative for CD45+CD14 which are coupled to the PE fluorophore

The origin (maternal/fetal) of the sorted cells is confirmed by STR analysis.

Five 60 mL samples were run:

| | Number of fetal | Number of fetal |
|---|---|---|
| | cells confirmed by | cells confirmed by |
| | STR | STR |
| Sample | ‘A’ samples | ‘B’ samples |
| 3460 | 0 | 0 |
| 3461 | 1 | 3 |
| 3476 | 3 | 2 |
| 3477 | 0 | 0 |
| 3478 | 0 | 0 |

1-84. (canceled)

85. A method of prenatal diagnostics comprising the steps of:
a. isolating a fetal amnion or chorion cell obtained from a maternal blood sample, wherein the maternal blood sample is from a woman between gestational ages 15 and 20 weeks, comprising the steps of:
i. contacting the cells comprised in the maternal blood sample with a ligand directed against a fetal amnion or chorion cell marker;
ii. enriching the maternal blood sample for fetal cells before or after step a.i;
iii. detecting the fetal amnion or chorion cell; and
iv. isolating the fetal amnion or chorion cell using FACS, or manual or automated cell picking;
b. diagnosing the fetus by analysing a genotype or a phenotype of the fetal amnion or chorion cell.

86. The method according to claim 85, wherein the fetal amnion or chorion cell marker is selected from IGF2, MUC16, UPK1B, EMP1, GPX8, FLT1/VEGFR1, RXFP1, CNR1, PRLR, THY1, NPR3/NPR-C and FERMT2 and combinations thereof.

87. The method according to claim 85, wherein the fetal amnion or chorion cell marker is for enriching in the step of enriching the maternal blood sample for fetal cells.

88. The method according to claim 87, wherein the fetal amnion or chorion cell marker for enriching is selected from IGF2, MUC16, UPK1B, EMP1, GPX8, FLT1/VEGFR1, RXFP1, CNR1, PRLR, NPR3/NPR-C, FERMT2 and THY1 and combinations thereof.

89. The method according to claim 85, wherein the step of enriching is followed by a step of staining.

90. The method according to claim 85, wherein the fetal amnion or chorion cell marker is used for staining a fetal amnion or chorion cell.

91. The method according to claim 90, wherein the fetal amnion or chorion cell marker for staining a fetal amnion or chorion cell is CK5, CK7, CK8, CK17, CK18, CK19, Ubiquitin, Pan CK or Vimentin and combinations thereof.

92. The method according to claim 85, wherein the method comprises a step of enriching fetal cells of the maternal blood sample and a step of staining fetal amnion or chorion cells.

93. The method according to claim 85, wherein the method further comprises a step of contacting the maternal blood sample with a ligand against maternal blood cells, wherein the ligand is against CD45, CD3, CD14, CD15, CD16 or CD19, and removing cells labelled with one or more of said ligands.

94. The method according to claim 93, wherein the ligand against maternal blood cells is used for discriminating between fetal cells and maternal blood cells.

95. The method according to claim 85, wherein the method further comprises a step of contacting the maternal blood sample with a fluorescent labelling agent directed against a nucleus.

96. The method according to claim 85, wherein the ligand directed against a fetal amnion or chorion cell marker is a magnetic or a fluorescent ligand.

97. The method according to claim 85, wherein the ligand directed against a fetal amnion or chorion cell marker is a ligand selected from antibodies, nucleotide probes, receptor ligands, and other specific binding molecules.

98. The method according to claim 85, wherein said enriching is done using magnetic activated cell sorting (MACS) or sorting on a fluorescence activated cell sorter (FACS).

99. The method according to claim 85, wherein a cellular fraction is separated from plasma of said maternal blood sample.

100. The method according to claim 99, wherein said cellular fraction is separated from said plasma by centrifugation.

101. The method according to claim 99, wherein said cellular fraction comprises both maternal cells and fetal cells.

102. The method according to claim 99, wherein said cellular fraction comprises red blood cells, white blood cells and fetal trophoblasts, fetal extravillous trophoblasts, fetal endovascular trophoblasts and fetal amnion or chorion cells.

103. The method according to claim 99, said method further comprising a step of fixating the blood cells subsequent to being separated from said plasma fraction.

104. A method of detecting a fetal amnion or chorion cell comprising the steps of:
a. contacting the cells comprised in a maternal blood sample obtained from a woman between the gestational ages 15 and 20 weeks with a ligand towards a fetal amnion or chorion cell marker;
b. enriching the cells comprised in said maternal blood sample before or after step a, and
c. and detecting the fetal amnion or chorion cell marker, thereby detecting the fetal amnion or chorion cell.
